(19) United States
(12) Patent Application Publication
(10) Pub. No.: US 2009/0149555 A1
(43) Pub. Date: Jun. 11, 2009

(54) METHOD FOR PREPARING POLYHYDROXYALKANOATES, POLYMERS THUS OBTAINED, COMPOSITIONS COMPRISING THEM AND THEIR USES

(76) Inventors: Jean-Francois Carpentier, Acigne (FR); Christophe Thomas, Paris (FR); Abderramane Amgoune, Saint-Etienne (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/093,850

(22) PCT Filed: Nov. 15, 2006
(86) PCT No.: PCT/EP06/68522

§ 371 Date: Sep. 25, 2008

(30) Foreign Application Priority Data
Nov. 15, 2005 (FR) 05 53471

(51) Int. Cl. A61K 47/34; C08G 63/82; C08L 67/04

(52) U.S. Cl. 514/772.3; 528/357; 525/415

(57) ABSTRACT

Method for preparing a polyhydroxyalkanoate (PHA) polymer by ring-opening polymerization of a lactone such as β-butyrolactone, that is preferably racemic, in which the polymerization is carried out in the presence of an initiator of formula (II):

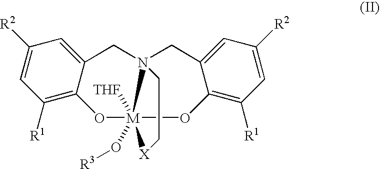

in which:
R³ represents a C₁₋₁₅ alkyl group such as a methyl, ethyl, isopropyl, n-propyl, n-butyl, isobutyl or tert-butyl group; or a benzyl group;
R¹ and R², being identical or different, each represent a group chosen from C₁₋₁₅ alkyl groups, such as methyl and tert-butyl groups; a cumyl group; an α,α-dimethylbenzyl group; an adamantyl group; a trityl group; and a trialkylsilyl group;
X represents O(R₄), S(R₄) or N(R₄)(R₅) where R₄ and R₅ each independently represent a C₁₋₁₅ alkyl group such as a methyl or ethyl group; or a benzyl group;
M is a metal from group 3 of the Periodic Table of the Elements such as Y, La or Nd.

Polymers obtainable by this method, compositions using them and the use of these polymers and compositions.

The present invention relates to a method for preparing polyhydroxyalkanoates (PHAs), in particular poly(3-hydroxybutyrate) (PHB).

More specifically, the invention relates to a method for preparing polyhydroxyalkanoates, such as poly(3-hydroxybutyrate) (PHB) by ring-opening polymerization of lactones, such as β-butyrolactone (BBL).

The invention also relates to PHA polymers capable of being obtained (obtainable) by this method, the compositions comprising these polymers, and the uses of these polymers and compositions.

The technical field of the invention may be very broadly defined as that of biodegradable polymers and their preparation.

Much interest has recently been taken in biodegradable polymers in order to replace conventional synthetic materials [1]. Among the novel biodegradable polymers that have been developed during the last ten years, polyhydroxyalkanoates (PHAs) are particularly advantageous. The properties of PHAs range from rigid to elastic, as a function of the length of the side chains or of the type of copolymer.

Furthermore, these materials combine the barrier film properties of polyesters with the good mechanical properties of polyethylene and of polypropylene prepared from oil.

The most common PHA is poly(3-hydroxybutyrate) (PHB) which is an aliphatic polyester produced by bacteria and other living organisms [2], [3]. This natural biodegradable and biocompatible polymer is isotactic with all the stereocentres in the (R) configuration [4], [5].

In document [6], bacteria which naturally produce polymers are used and their metabolism is converted so that they produce biodegradable polymers and copolymers of PHB, from plant sugars or plant oils.

The PHB produced by bacteria and isolated is highly crystalline, is in the enantiomerically pure form where all the stereocentres are in the (R) configuration, melts at 175-180° C., with a glass transition temperature (T_{g}) of 9° C. [7].

However, the highly crystalline nature and the low thermal stability of PHB are the source of difficulties during its treatment in the melt state, as its temperature of degradation (which gives rise to crotonate groups) begins immediately above the melting point (Tₘ), which limits its industrial importance.

To improve the treatability (processability), a syndiotactic PHB, alternately containing blocks formed from competing (R) and (S) β-hydroxybutyrate units, could be an advantageous alternative as a biodegradable industrial plastic, on condition that its melting point is below that of the isotactic polyester.

Numerous methods exist for the synthesis of PHAs, such as PHB, but the easiest and most promising is the ring-opening polymerization (ROP) of lactones, such as β-butyrolactone, where the driving force of the polymerization is the relaxation of the ring strain [8].

It has been shown that a highly isotactic (R or S) polyhydroxybutyrate could be obtained when optically pure (R), respectively (S) β-butyrolactone was used [9], whereas when a racemic mixture of (R) and (S) β-butyrolactone is used, atactic PHB [10] and PHB enriched with isotactic [11] or syndiotactic [12] diads may be formed.

Isotactic, atactic or syndiotactic microstructures have been able to be observed during the polymerization from racemic BBL using metallic initiators.

However, apart from the recently mentioned distannoxane [12a] and alkylzinc alcoholate [10c] catalysts, most of the systems studied by ring-opening polymerization of BBL are extremely slow and/or are not capable of producing a PHB having a high molecular weight in a controlled manner. In a recent document, Coates et al. [13], described that β-diiminate zinc alkoxide complexes are capable of polymerizing BBL with activities that have not been achieved until then, under mild conditions, in order to prepare PHAs in a controlled manner. High molecular weights were achieved at ambient temperature with a relatively good polydispersity (PDI=1.20) after 12 h, but all the PHBs obtained were atactic.

Disclosed recently have been several single-site, well-defined group 3 metal bis(phenolate) complexes which have an effective action as initiators in the synthesis of biodegradable polymers, for example in the preparation of heterotactic and syndiotactic polylactic acid from racemic lactide and meso-lactide respectively [14].

Since the PHAs produced by bacterial synthesis remain, despite many potential uses, too expensive for widespread use, there is, with regard to what has been mentioned previously, a need for a preparation method via chemical synthesis which makes it possible to prepare PHAs such as PHB, especially syndiotactic PHAs with a narrow polydispersity and a high molecular weight, rapidly, selectively, and with a high yield.

The objective of the present invention is to provide a method for preparing polyalkoxyalkanoates PHAs, and in particular PHB, which meet, amongst other things, these needs.

The objective of the present invention is also to provide such a preparation method which does not have the drawbacks, defects, limitations and disadvantages of the methods of the prior art and which solves the problems of the methods of the prior art.

This objective and others too are achieved, according to the invention, by a method for preparing a polyhydroxyalkanoate (PHA) polymer by ring-opening polymerization of a lactone of formula (I):

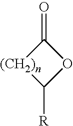

where n is an integer from 1 to 4, and R represents an hydrogen or a linear or branched C₁₋₄ alkyl group, characterized in that the polymerization is carried out in the presence of an initiator of formula (II):

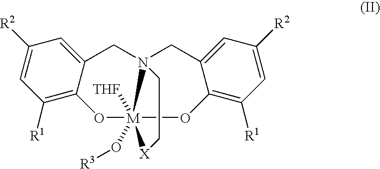

in which:
R³ represents a C₁₋₁₅ alkyl group such as a methyl, ethyl, isopropyl, n-propyl, n-butyl, isobutyl or tert-butyl group; or a benzyl group;
R¹ and R², being identical or different, each represent a group chosen from C₁₋₁₅ alkyl groups, such as a methyl group; a tert-butyl group; a cumyl group; an α,α-dimethylbenzyl group; an adamantyl group; a trityl group; and a (C₃ to C₁₅)trialkylsilyl group;
X represents O(R₄), S(R₄) or N(R₄)(R₅) where R₄ and R₅ each independently represent a C₁₋₁₅ alkyl group such as a methyl or ethyl group; a benzyl group;
M is a metal from group 3 of the Periodic Table of the Elements such as Y, La or Nd.

The lactone is generally chosen from the lactones corresponding to the following formulae:

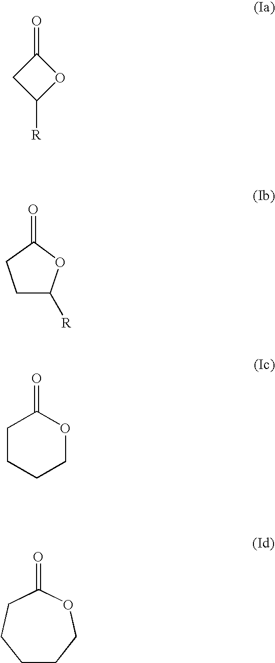

in which R has the meaning already given above. Preferably, in the formula (Ia), R represents H, or a methyl or ethyl group and preferably in the formula (Ib), R represents H, or a methyl, ethyl or propyl group.

The lactone with which the polymerization is preferably carried out is β-butyrolactone, especially racemic β-butyrolactone.

The lactone that is polymerized is preferably a racemic lactone when its structure allows it, that is to say when, in the formulae II above, R is other than H. The lactone could thus be racemic β-butyrolactone or racemic γ-valerolactone. The method according to the invention then makes it possible to obtain, very preferably, the syndiotactic PHA (for example, PHB) polymer with a degree of syndiotacticity generally from 70 to 99%.

The Tₘ of such polymers generally varies from 130 to 180° C. depending on the syndiotacticity.

The initiator of formula (II) is generally chosen from the compounds of formulae below:

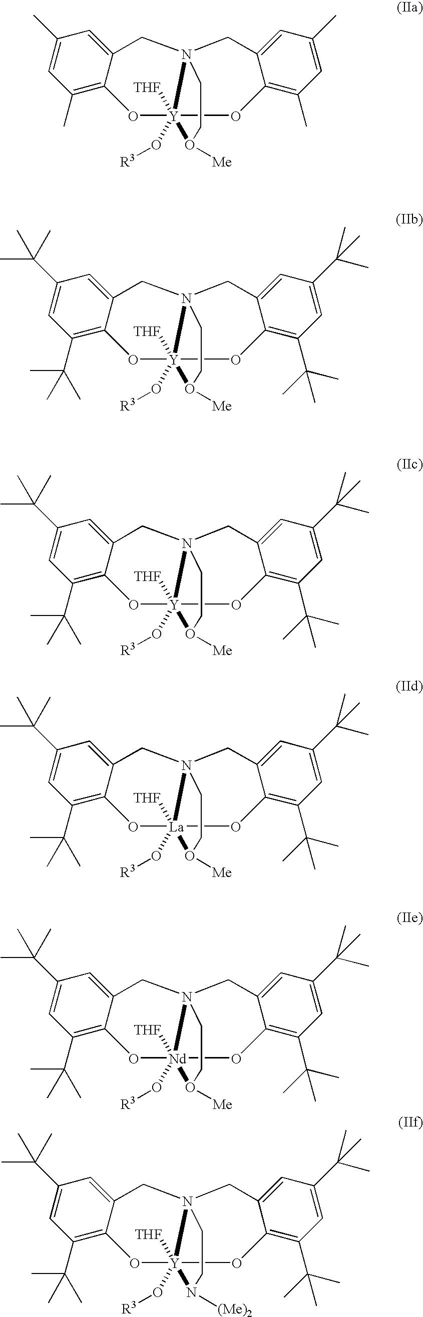

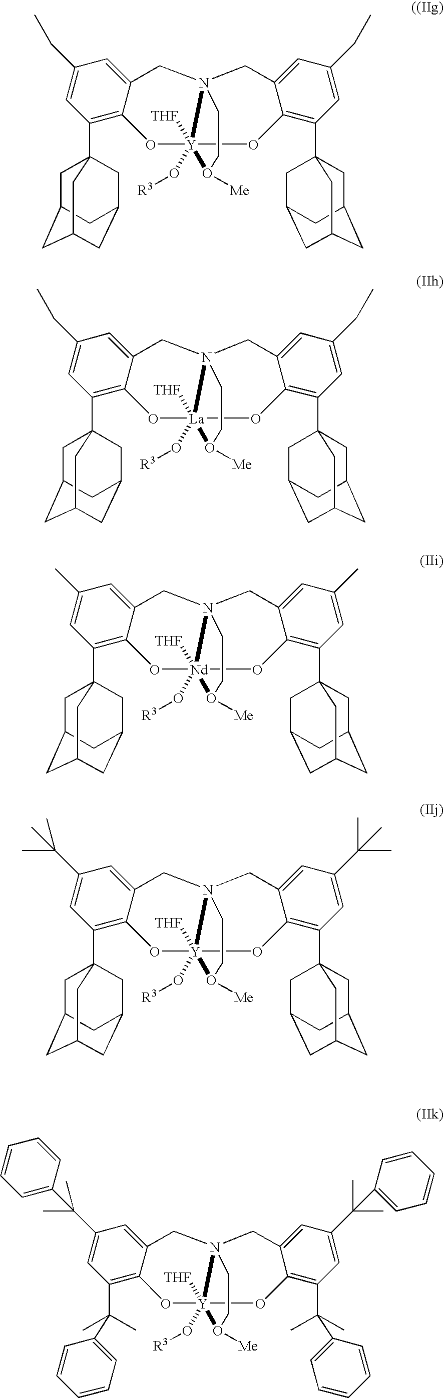

The preferred initiator of formula (II) is the following compound:

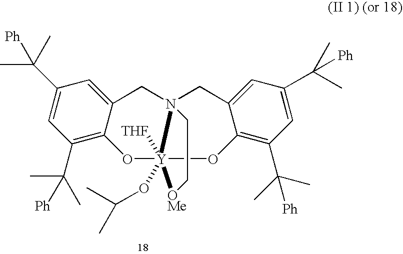

The polymerization is generally carried out in a solvent chosen from, for example, toluene, tetrahydro-furan (THF), chlorobenzene and mixtures thereof.

The preferred solvent is toluene.

The ratio of the lactone concentration to that of the initiator is generally from 50 to 5000, preferably from 100 to 2000.

The polymerization is generally carried out at a temperature from −30 to 120° C., preferably from 0 to 60° C., more preferably from 15 to 30° C., for example at 20° C.

The method according to the invention applies, in particular, to the polymerization of racemic β-butyro-lactone, carried out with the complex of formula (III), in toluene, at a temperature from −30° C. to 110° C., preferably from 0 to 60° C., and the ratio of the β-butyrolactone concentration to that of the initiator (of the complex) is generally from 100 to 2000.

The polymer prepared generally has a number-average molecular weight of 4000 to 500 000, preferably from 8000 or 9000 to 170 000.

Moreover, the polymer generally has a polydispersity index PDI from 1.01 to 1.50, preferably from 1.05 to 1.20.

The method according to the invention is fundamentally different from the methods of the prior art since it uses, for the ring-opening polymerization of lactones, specific initiator compounds which have never been used in the ring-opening polymerization of lactones, and in that it results in PHAs having a high syndiotactic content.

Some of these initiators have already been used for the ring-opening polymerization of lactides, but there was no reason to assume that they could also be used successfully in the polymerization of lactones. This is because the initiators are generally specific to the ring-opening polymerization of one class of specific compounds and the excellent results obtained with certain initiators for the polymerization of lactides was on no account reason to predict that such initiators would be as advantageous within the context of the polymerization of lactones.

The method according to the invention makes it possible to obtain, in a perfectly controlled manner with a very high activity, a very high productivity, a very high yield, and a very high selectivity of the PHA polymers, of high molecular weight and with a narrow molecular weight distribution.

In particular, heterotactic PHA polymers with a narrow molecular weight distribution, said molecular weight being a high number-average molecular weight, for example from 4000 to 500 000, have been obtained thanks to the method of the invention, with very high or even quantitative activities and productivities at ambient temperature.

The method according to the invention makes it possible, in particular, to control the tacticity of the polymer obtained and to preferably prepare predominantly syndiotactic polymers from racemic lactones when these lactones exist.

The invention additionally relates to a polyhydroxy-alkanoate polymer capable of being obtained (obtainable) by the method such as described above.

This polyhydroxyalkanoate polymer is advantageously a syndiotactic PHA capable of being obtained (obtainable) by polymerization of a racemic lactone (I).

Said lactone is preferably racemic β-butyrolactone or racemic γ-valerolactone.

The polymer capable of being obtained by the method of the invention, when it is syndiotactic, generally has a degree of syndiotacticity of 70 to 99%.

The polymer capable of being obtained by the method of the invention generally has a number-average molecular weight from 4000 to 500 000, preferably from 8000 or 9000 to 170 000.

The polymer capable of being obtained by the method of the invention generally has a polydispersity index PDI from 1.01 to 1.50, preferably from 1.05 to 1.20.

The invention also relates to a material composition comprising the polyhydroxyalkanoate polymer described above and other ingredients.

Advantageously, this composition is biodegradable; in which case, this composition generally comprises, in addition, one or more biodegradable polymers different from the polyhydroxyalkanoate.

In the compositions according to the invention, the polyhydroxyalkanoate polymer is generally present in an amount of 0.1 to 99.9%, preferably 1 to 99%, more preferably 5 to 90%, better 10 to 80%, better still 20 to 70%, for example 40 to 60%, especially 50% or 55% by weight of the total weight of the composition.

The invention also relates to the use of the polymer capable of being prepared by the method of the invention or of the composition such as described above in packaging, especially food packaging.

The invention also relates to the use of the polymer capable of being prepared by the method of the invention or of the composition such as described above in biomedical devices and processes or for biomedical applications.

The invention also relates to the use of the polymer capable of being prepared by the method of the invention or of the composition such as described above in surgical fasteners such as sutures, agrafes and plates.

The invention finally relates to the use of the polymer capable of being prepared by the method of the invention or of the composition such as described above for the controlled, delayed, release of medicaments (drugs).

The invention will now be described in detail in the description which follows, given by way of illustration and non-limitingly, in connection with the appended drawings in which:

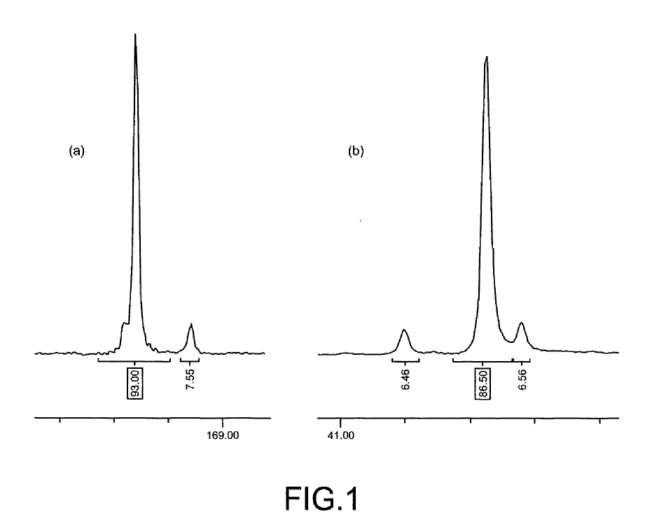

FIG. 1 represents the carbonyl (a) and methylene (b) regions of the ¹³C NMR spectra (125 MHz, CDCl₃) of PHB prepared by polymerization of racemic β-butyrolactone with the complex (III); and

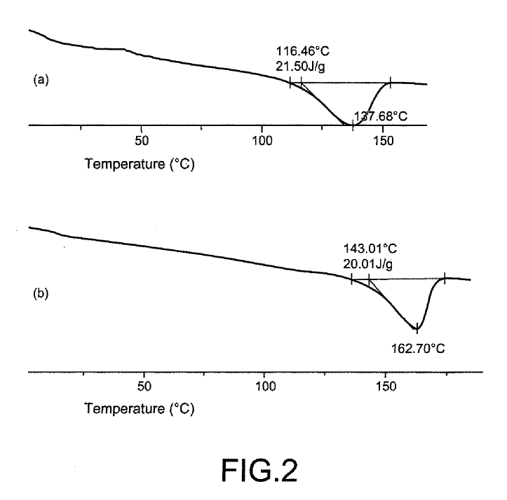

FIG. 2 represents the thermograms obtained by differential scanning calorimetry analysis respectively of an 85% syndiotactic PHB polymer (Example 12, curve a)) and of a 91% syndiotactic PHB polymer (Example 2, curve b)).

In the description which follows, the method according to the invention is described by predominantly referring to the polymerization of racemic β-butyro-lactone preferably by the complex of formula (III), but it is very obvious that a person skilled in the art will understand that this description may also apply to the polymerization of other racemic or non-racemic lactones with the same complex or with other complexes described in the present document.

The initiator complexes used in the method according to the invention are prepared from diamino and alkoxyamino bisphenol H₂L ligands, for example 1 to 6, which are generally synthesized by double Mannich condensations of the corresponding substituted phenol, of formaldehyde and of the appropriate alkoxyamine or diamine according to scheme 1 below:

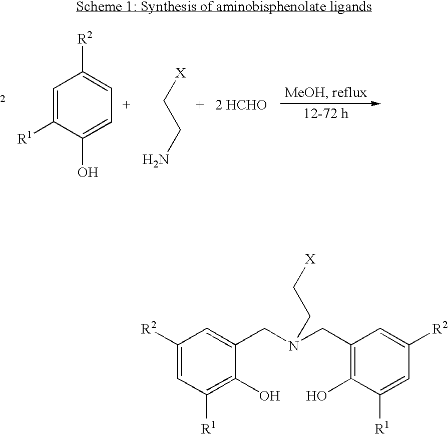

In scheme 1, R¹, R² and X have the meaning already specified above.

They may be isolated with average to moderate yields in the form of microcrystalline powders. Some of these N,N-substituted compounds are difficult to obtain due to the formation of benzoxazines as by-products which takes place by ring-closure of the intermediate N-substituted product. Given below in scheme 2 is the formula of particular aminobisphenolate ligands 1 to 6.

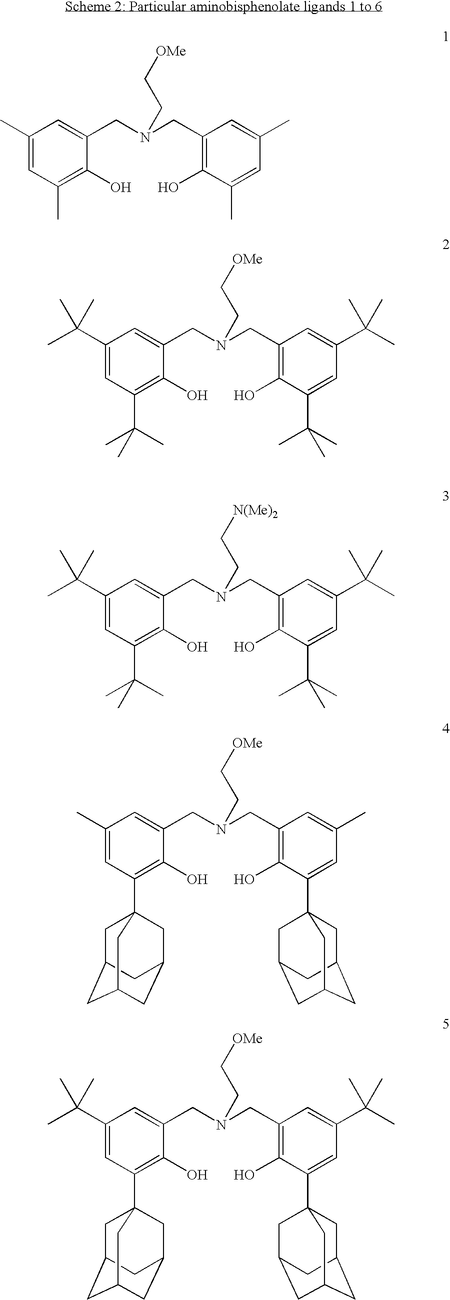

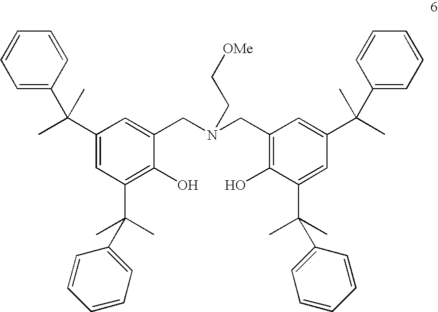

Group 3 metal precursors [MR′₃(THF)₂] with M=Y, La; and R′=CH₂SiMe₃, N(SiHMe₂)₂ and [Na{N(SiMe₃)₂}₃], are each treated with one equivalent of bisphenolate ligand (the general formula appears in scheme 1), for example with one equivalent of each of the H₂L bisphenols 1 to 6 to give the yttrium and lanthanum complexes [(L)M(R′)(THF)] 7 to 10, 12 to 14, 16 and 17, and [(L)Nd.{N(SiMe₃)₂}] 11 and 15 with good yields (scheme 3).

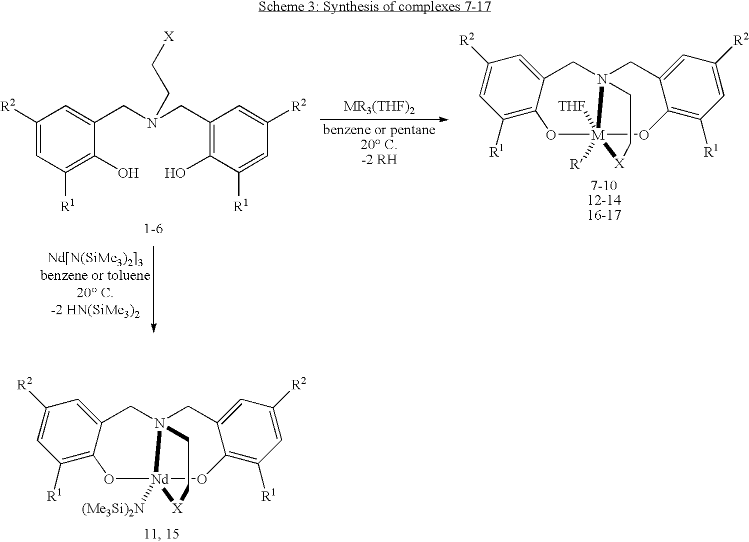

Table 1 gives the meanings of the ligands, of the metal and of the substituents for the complexes 7 to 17.

TABLE 1

| Complex | Metal | Ligand | R′ |
|---|---|---|---|
| 7 | Y | 1 | N(SiMe₃)₂ |
| 8 | Y | 2 | N(SiHMe₂)₂ |
| 9 | Y | 2 | CH₂SiMe₃ |
| 10 | La | 2 | N(SiHMe₂)₂ |
| 11 | Nd | 2 | N(SiMe₃)₂ |
| 12 | Y | 3 | N(SiHMe₂)₂ |
| 13 | Y | 4 | N(SiHMe₂)₂ |
| 14 | La | 4 | N(SiHMe₂)₂ |
| 15 | Nd | 4 | N(SiMe₃)₂ |
| 16 | Y | 5 | N(SiHMe₂)₂ |
| 17 | Y | 6 | N(SiHMe₂)₂ |

The complexes used in the method according to the invention such as the complex (III) are prepared from, for example, complexes 7 to 17 by reaction of these complexes with the appropriate alcohol of formula R³—OH (where R³ has the meaning already given above) at ambient temperature in a solvent such as benzene-d₆ or THF-d₈ (scheme 4). A preferred alcohol is isopropanol.

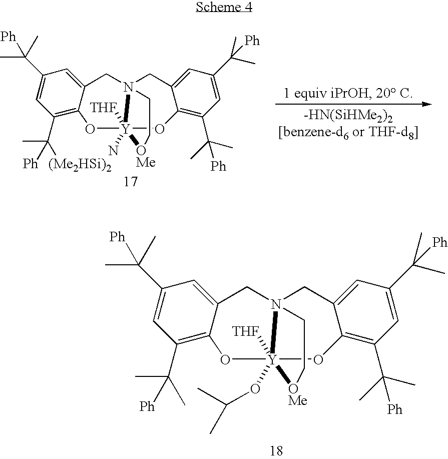

The invention will now be described in the experimental section and the following examples which relate to the synthesis of aminobisphenoxy complexes and to their trial in the polymerization of racemic β-butyrolactone.

### General Conditions

The synthesis of the complexes and the polymerization tests were carried out in a glovebox. The glassware used was dried in an oven at 120° C. overnight and cooled under vacuum before use. The solvents used (toluene, THF, pentane) were distilled over Na/K under argon and were degassed several times before use. The benzene-d₆ was dried and degassed before use. The metallic precursors Y(N(SiHMe₂)₂)₃THF₂, La(N(SiHMe₂)₂)₃THF₂, Nd(N(TMS)₂)₃ were synthesized in the laboratory. The racemic butyrolactone was distilled twice over CaH₂ under vacuum, then was kept in a glovebox. The ligands were synthesized from commercial reactants, or from reactants synthesized previously in the laboratory. The ligands 2 and 3 were synthesized according to the same procedures already published.

### Synthesis of the aminobis(2,4-dimethylphenoxy) ligand (Compound 1)

Formaldehyde (1.2 ml, 16 mmol) and methoxyethylamine (0.52 ml, 6 mmol) were mixed at ambient temperature. The mixture was then added to a solution of 2,4-dimethylphenol (1.95 g, 16 mmol) in methanol (4 ml). The reaction medium was then refluxed for 24 h. After the mixture had been cooled, a brown precipitate was observed, the solution was separated from the precipitate and this precipitate was redissolved in a minimum amount of methanol, then refluxed for 2 h. A white precipitate was thus formed at the bottom of the solution. The mixture was filtered under vacuum, then the white powder obtained was dried under vacuum. The filtrate was left in a refrigerator for 24 h, and white crystals were obtained (1.1 g, yield 52%). ¹H NMR (200 MHz, CDCl₃, 25° C.): δ 8.35 (s, 2H; ArOH), 6.85 (s, 2H; ArH), 6.67 (s, 2H; ArH), 3.72 (s, 4H; ArCH₂), 3.58 (t, ³J(H,H)=5.1 Hz, 2H; NCH₂CH₂O), 3.47 (s, 3H; OCH₃), 2.70 (t, ³J(H,H)=4.9 Hz, 2H; NCH₂CH₂O), 2.20 (s, 12H; CH₃); ¹³C{¹H} NMR (75 MHz, benzene-d₆, 25° C.): δ 152.84, 131.37, 121.24 (A-Cq), 127.68, 127.36, 125.15 (Ar—CH), 70.89 (NCH₂CH₂O), 58.17 (OCH₃), 57.04 (NCH₂CH₂O), 50.77 (CH₂Ar), 20.24, 16.03 (CH₃); HRMS (70 eV, EI): m/z calculated for C₂₁H₂₉N₁O₃: 343.2147; found: 343.2139 [M⁺].

### Synthesis of the aminobis(2-adamantyl-4-methylphenoxy) ligand (Compound 4)

Formaldehyde (0.12 ml, 1.60 mmol) and methoxyethylamine (0.052 ml, 0.60 mmol) were mixed at ambient temperature. The mixture was then added to a solution of 2-adamantyl-4-methylphenol (0.38 g, 1.60 mmol) in methanol (1.5 ml). The reaction medium was then refluxed for 48 h. After the mixture had been cooled, a brown precipitate was observed, the solution was separated from the precipitate and this precipitate was redissolved in a minimum amount of methanol, then refluxed for 2 h. A white precipitate was thus formed at the bottom of the solution. The mixture was filtered under vacuum, then the white powder obtained was dried under vacuum (0.22 g, yield 62%). ¹H NMR (CDCl₃, 200 MHz): δ 8.37 (s, 2H; ArOH), 6.93 (br s, 2H; ArH), 6.69 (br s, 2H; ArH), 3.67 (s, 4H; ArCH₂), 3.48 (br t, ³J(H,H)=5.1 Hz, 2H; NCH₂CH₂O), 3.44 (s, 3H; OCH₃), 2.74 (t, ³J(H,H)=5.1 Hz, 2H; NCH₂CH₂O), 2.22 (s, 6H; CH₃), 2.15 (s, 12H; CH₂-adamantyl), 2.04 (s, 6H; CH-adamantyl), 1.76 (s, 12H; CH₂-adamantyl); ¹³C{¹H} NMR (75 MHz, benzene-d₆, 25° C.): δ 153.61, 137.39 (A-Cq), 128.69, 122.71 (Ar—CH), 71.07 (NCH₂CH₂O), 58.13 (OCH₃), 57.44 (CH₂Ar), 51.28 (NCH₂CH₂O), 40.73 (adamantyl), 37.44 (adamantyl), 37.13 (adamantyl), 29.60 (adamantyl), 20.83 (CH₃); HRMS (4000 V, ESI): m/z calculated for C₃₉H₅₄N₁O₃: 584.4103. found: 584.4107 [M⁺+H].

### Synthesis of 2-adamantyl-4-tert-butylphenol

In a round-bottomed flask under argon, 4-tert-butylphenol (4 g; 0.026 mol) was dissolved in a xylene/DMF (20 ml/10 ml) mixture, then sodium cut into fine chips (0.5 g; 0.026 mol) was added under argon. Once the sodium had completely dissolved (yellow coloration of the solution), 1-chloroadamantane (4.55 g; 0.026 mol) was added. The mixture continued to be stirred at 85° C. for 24 h. After having left the reaction medium to return to ambient temperature, the reaction mixture was dissolved in 100 ml of ether, then the aqueous phase was extracted with 100 ml of a 10% KOH solution. The aqueous phase was washed with 3 times 100 ml of ether. The etherated phases were combined, then washed with 100 ml of water and dried over MgSO₄. The solvent was evaporated, and the oil obtained was dried under vacuum. 4.85 g of crude product were obtained (yield 65%), the product was purified by column chromatography to obtain 1.84 g of pure 2-adamantyl-4-tert-butylphenol (yield 26%). ¹H NMR (CDCl₃, 200 MHz): δ 7.25 (s, 1H, phenyl), 7.09 (dd, J=8 Hz, 1H, phenyl), 6.59 (d, J=8.1 Hz, 1H, phenyl), 4.59 (s, 1H, PhOH), 2.13 (bs, 9H, adamantyl), 1.78 (s, 6H, adamantyl), 1.29 (s, 9H, t-Bu).

### Synthesis of the aminobis(2-adamantyl-4-tert-butyl-phenoxy) ligand (Compound 5)

Formaldehyde (0.17 ml, 1.75 mmol) and methoxyethylamine (0.057 ml, 0.65 mmol) were mixed at ambient temperature. The mixture was then added to a solution of 2-adamantyl-4-tert-butylphenol (0.5 g, 1.75 mmol) in methanol (2 ml). The reaction medium was then refluxed for between 24 and 48 h. After the mixture had been cooled, a brown precipitate was observed, the solution was separated from the precipitate and this precipitate was redissolved in a minimum amount of methanol, then refluxed for 2 h. A white precipitate was thus formed at the bottom of the solution. The mixture was filtered under vacuum, then the white powder obtained was dried under vacuum (0.13 g, yield 30%). The filtrate was left in the fridge for 24 h, and white crystals were obtained (yield 56%), corresponding to benzoxazine (2). When the benzoxazine obtained was mixed with 1 equivalent of 2-adamantyl-4-tert-butylphenol and one equivalent of formaldehyde, then refluxed for 24 h, the desired product was formed (52%). ¹H NMR (C₆D₆, 200 MHz): δ 8.80 (s, 2H; PhOH), 7.48 (d, ⁴J(H,H)=2.2 Hz, 2H; ArH), 6.96 (d, ⁴J(H,H)=2.2 Hz, 2H; 2H; ArH), 3.53 (s, 4H; ArCH₂), 2.99 (br s, 5H; OCH₃+NCH₂CH₂O), 2.50 (s, 12H; CH₂ adamantyl), 2.35 (t, ³J(H,H)=2.2 Hz, 2H; NCH₂CH₂O), 2.18 (s, 6H; CH adamantyl), 1.92 (br m, 12H; CH₂ adamantyl), 1.37 (s, 18H; C(CH₃)₃; ¹³C{¹H} NMR (75 MHz, benzene-d₆, 25° C.): δ 153.39 140.94, 136.59, 122.07 (Cq Ar), 124.76, 123.36 (Ar), 70.79 (NCH₂CH₂O), 58.12 (OCH₃), 57.63 (CH₂Ar), 50.93 (NCH₂CH₂O), 40.70 (adamantyl), 37.37 (adamantyl), 37.33 (adamantyl), 34.07 (adamantyl), 31.65 (C(CH₃)₃), 29.51 (C(CH₃)₃); HRMS (4000 V, ESI): m/z calculated for C₄₅H₆₆N₁O₃: 668.5042. found: 668.5037 [M⁺+H].

### Synthesis of the aminobis(2,4-dicumylphenoxy) ligand (Compound 6)

Formaldehyde (1.12 ml, 15.1 mmol) and methoxyethylamine (0.5 ml, 5.7 mmol) were mixed at ambient temperature. The mixture was then added to a solution of 2,4-dicumylphenol (5 g, 15.1 mmol) in methanol (4 ml). The reaction medium was then refluxed for 72 h. After the mixture had been cooled, a white precipitate was observed, the solution was separated from the precipitate and this precipitate was redissolved in a minimum amount of methanol, then refluxed for 24 h. A white precipitate was thus formed at the bottom of the solution. The mixture was filtered under vacuum, then the white powder obtained was dried under vacuum (yield 30%). ¹H NMR (C₆D₆, 200 MHz): δ 8.11 (s, 2H; PhOH), 7.47 (d, ⁴J(H,H)=1.8 Hz, 2H; ArH), 7.33 (t, ³J(H,H)=7.7 Hz, 16H; phenylcumyl), 7.06 (m, 4H; phenylcumyl), 6.83 (d, ³J(H,H)=1.3 Hz, 2H; ArH), 3.28 (s, 4H; ArCH₂), 2.68 (br s, 5H; OCH₃+NCH₂CH₂O), 2.15 (t, ³J(H,H)=5.1 Hz, 2H; NCH₂CH₂O), 1.75 (s, 12H; CH₃ cumyl), 1.68 (s, 12H; CH₃ cumyl); ¹³C{¹H} NMR (75 MHz, benzene-d₆, 25° C.): δ 153.52, 152.16, 151.70, 141.29, 136.51 (Cq Ar), 128.96, 128.40, 127.63, 126.80, 126.32, 123.57 (Ar), 71.57 (s, NCH₂CH₂O) 58.68 (OCH₃), 57.60 (CH₂Ar), 51.67 (s, NCH₂CH₂O), 43.26 (C(CH₃)₂), 42.95 (C(CH₃)₂), 31.82 (CH₃), 30.17 (CH₃); HRMS (4000 V, ESI: m/z calculated for C₅₃H₆₂N₁O₃: 760.4729. found: 760.4726 [M⁺+H]

Reaction of Y[N(SiMe₃)₂]₃ with 1. Generation of “7”

A solution of 1 (53.4 mg, 0.105 mmol) in toluene (5 ml) was added to a solution of Y[N(SiMe₃)₂]₃ (60.0 mg, 0.105 mmol) in toluene (5 ml) at ambient temperature. The mixture was stirred for 12 h at ambient temperature and 2 h at 60° C. Then, the solution was evaporated under vacuum. The solid was washed with a small amount of cold pentane, then dried under vacuum to give a white powder (43.9 mg, 67%). ¹H NMR (200 MHz, benzene-d₆, 25° C.) characteristic peaks: δ 5.60 (d, ²J(H,H)=11.7 Hz, 0.5H; ArCH₂), 5.45 (d, ²J(H,H)=11.9 Hz, 0.5H; ArCH₂), 5.22 (d, J(H,H)=12.4 Hz, 0.8H; ArCH₂), 5.09 (dd, ²J(H,H)=4.0 Hz, ²J(H,H)=11.3 Hz, 0.8H; ArCH₂), 4.71 (d, ²J(H,H)=12.4 Hz, 2H; ArCH₂), 4.61 (d, ²J(H,H)=12.4 Hz, 2H; ArCH₂), 4.03 (d, ²J(H,H)=11.9 Hz, 0.6H; ArCH₂), 3.58 (d, ²J(H,H) 12.4 Hz, 0.6H; ArCH₂), 0.37 (br s, 18H; NSi(CH₃)₃), 0.31 (s, 16H; NSi(CH₃)₃). The peaks for HNSi(CH₃)₃ were also observed (δ 0.12 (s, 92H)), whereas the ArOH peak disappeared. The complex was used directly in catalysis.

### Synthesis of Complex 8

A solution of 2 (0.153 g, 0.30 mmol) in pentane (5 ml) was added to a solution of Y[N(SiHMe₂)₂]₃(THF)₂ (0.189 g, 0.30 mmol) in pentane (5 ml) at ambient temperature. The mixture was stirred for 12 h at ambient temperature and a white precipitate was then obtained. In order to ensure a complete conversion, the mixture was again left stirring for a further 10 h. The solid was then filtered to give a white powder 8 (0.163 g, 67%). ¹H NMR (300 MHz, benzene-d₆, 25° C.): δ 7.60 (d, ⁴J(H,H)=2.5 Hz, 2H; ArH), 7.10 (d, ⁴J(H,H)=2.3 Hz, 2H; ArH), 5.14 (m, 2H; SiH), 3.87 (d, J(H,H)=12.5 Hz, 2H; overlapping with the signal of the THF, ArCH₂), 3.84 (br m, 4H; α-CH₂ THF), 2.97 (d, ²J(H,H)=12.5 Hz, 2H; ArCH₂), 2.84 (s, 3H; OCH₃), 2.71 (t, ³J(H,H)=5.2 Hz, 2H; NCH₂CH₂O), 2.31 (t, ³J(H,H)=5.2 Hz, 2H; NCH₂CH₂O), 1.79 (s, 18H; C(CH₃)₃), 1.47 (s, 18H; C(CH₃)₃), 1.18 (m, 4H; β-CH₂ THF), 0.49 (d, ⁴J(H,H)=3.0 Hz, 12H; HSi(CH₃)₂); ¹H NMR (300 MHz, THF-d₈, 25° C.): δ 7.19 (d, ⁴J(H,H)=2.5 Hz, 2H; ArH), 6.91 (br s, 2H; ArH), 4.90 (m, 2H; SiH), 4.00 (d, ²J(H,H)=12.5 Hz, 2H; ArCH₂), 3.58 (m, 4H; α-CH₂ THF), overlapping with the resonances of the solvent), 3.26 (m, 7H; ArCH₂, OCH₃, NCH₂CH₂O), 2.70 (t, ³J(H,H)=5.2 Hz, 2H; NCH₂CH₂O), 1.73 (m, 4H; β-CH₂ THF, overlapping with the resonances of the solvent), 1.48 (s, 18H; C(CH₃)₃), 1.26 (s, 18H; C(CH₃)₃), 0.15 (d, ⁴J(H,H)=3.0 Hz, 12H; HSi(CH₃)₂); ¹³C{¹H} NMR (75 MHz, benzene-d₆, 25° C.): δ 161.38, 136.53, 125.40, 124.11 (Ar), 73.11 (NCH₂CH₂O), 70.96 (α-CH₂ THF), 64.54 (CH₂Ar), 60.43 (OCH₃), 49.57 (NCH₂CH₂O), 35.46 (C(CH₃)₃), 34.04 (C(CH₃)₃), 32.06 (C(CH₃)₃), 30.30 (C(CH₃)₃), 24.95 (s, β-CH₂ THF), 4.22 HSi(CH₃)₂; elemental analysis (%) calculated for C₄₁H₇₃N₂O₄Si₂Y: C, 61.32; H, 9.17; N, 3.49. found: C, 61.74; H, 9.36; N, 3.36.

### Synthesis of Complex 9

A solution of 2 (0.153 g, 0.30 mmol) in pentane (5 ml) was added to a solution of Y(CH₂SiMe₃)₃(THF)₂ (0.148 g, 0.30 mmol) in pentane (5 ml) at 0° C. The mixture was stirred for 2 h at 0° C. Then, the solution was evaporated under vacuum. The solid was washed with a small amount of cold pentane, then dried under vacuum to give a white powder 9 (0.16 g, 70%). ¹H NMR (300 MHz, benzene-d₆, 25° C.): δ 7.59 (d, ⁴J(H,H)=2.5 Hz, 2H; ArH), 7.08 (d, ⁴J(H,H)=2.5 Hz, 2H; ArH), 3.87 (br m, 4H; α-CH₂ THF), 3.76 (d, ²J(H,H)=12.5 Hz, 2H; ArCH₂), 2.92 (d, ²J(H,H)=12.5 Hz, 2H; ArCH₂), 2.88 (s, 3H; OCH₃), 2.44 (t, ³J(H,H)=5.3 Hz, 2H; (NCH₂CH₂O), 2.21 (t, ³J(H,H)=5.3 Hz, 2H; (NCH₂CH₂O), 1.80 (s, 18H; C(CH₃)₃), 1.46 (s, 18H; C(CH₃)₃), 1.27 (br m, 4H; β-CH₂ THF), 0.49 (s, 9H; Si(CH₃)₃), 0.40 (d, ²J(Y—H)=3.1 Hz, 2H; CH₂Si(CH₃)₃; ¹³C{H} NMR (75 MHz, benzene-d₆, 25° C.): δ 161.38, 136.59, 136.40, 125.40, 124.21, 123.90 (Ar), 73.84 (NCH₂CH₂O), 70.66 (α-CH₂ THF), 64.65 (CH₂Ar), 61.09 (OCH₃), 49.09 (NCH₂CH₂O), 35.39 (C(CH₃)₃), 34.04 (C(CH₃)₃), 32.05 (C(CH₃)₃), 30.11 (C(CH₃)₃), 24.92 (β-CH₂ THF), 24.70 (d, ¹J(C—Y)=46.4 Hz; YCH₂), 4.64 (Si(CH₃)₃); elemental analysis (%) calculated for C₄₁H₇₀NO₄SiY: C, 64.97; H, 9.31; N, 1.85. found: C, 65.11; H, 9.65; N, 1.74.

### Synthesis of Complex 10

A solution of 2 (0.204 g, 0.40 mmol) in pentane (5 ml) was added to a solution of La[N(SiHMe₂)₂]₃(THF)₂ (0.272 g, 0.40 mmol) in pentane (5 ml) at ambient temperature. The mixture was stirred for 24 h at ambient temperature, then the solution was evaporated under vacuum. The solid was washed with a small amount of cold pentane, then dried under vacuum to give a white powder 10 (0.18 g, 92%). ¹H NMR (300 MHz, benzene-d₆, 25° C.): δ 7.57 (d, ⁴J(H,H)=2.3 Hz, 2H; ArH), 7.11 (d, ⁴J(H,H)=2.5 Hz, 2H; ArH), 5.25 (m, 2H; SiH), 3.76 (br m, 4H; α-CH₂ THF), 3.58 (d, ²J(H,H)=12.5 Hz, 2H; ArCH₂), 3.32 (d, ²J(H,H)=12.5 Hz, 2H; ArCH₂), 3.07 (s, 3H; OCH₃), 2.77 (t, ³J(H,H)=5.2 Hz, 2H; (NCH₂CH₂O), 2.27 (m, 2H; (NCH₂CH₂O), 1.73 (s, 18H; C(CH₃)₃), 1.44 (s, 18H; C(CH₃)₃), 1.22 (m, 4H; β-CH₂ THF), 0.48 (d, ⁴J(H,H)=3.0 Hz, 12H; Si(CH₃)₂); ¹³C{¹H} NMR (75 MHz, benzene-d₆, 25° C.): δ 161.8, 136.6, 135.6, 125.8, 124.3, 123.9 (Ar), 71.9 (NCH₂CH₂O), 69.9 (α-CH₂ THF), 61.6 (CH₂Ar), 60.9 (OCH₃), 50.7 (NCH₂CH₂O), 35.3 (C(CH₃)₃), 34.0 (C(CH₃)₃), 32.0 (C(CH₃)₃), 30.1 C(CH₃)₃), 25.0 (β-CH₂ THF), 3.5 (Si(CH₃)₂; elemental analysis (%) calculated for C₄₁H₇₃N₂O₄LaSi₂: C, 57.72; H, 8.62; N, 3.28. found: C, 57.91; H, 9.03; N, 3.18.

### Synthesis of Complex 11

The neodymium amide Nd(N(SiMe₃)₂)₃ (10 mg; 0.016 mmol) was dissolved in deuterated benzene (2 ml) in an NMR tube, then one equivalent of ligand 2 (9.3 mg; 0.016 mmol) was added. The solution was left overnight at ambient temperature. Then the solution was raised to 60° C. for several hours. Once the solvent had evaporated, the complex was directly used in catalysis.

Reaction of Y[N(SiHMe₂)₂]₃(THF)₂ with 3 in an NMR Tube. Generation of 12.

Added at ambient temperature to a solution of Y[N(SiHMe₂)₂]₃(THF)₂ (17.1 mg, 0.030 mmol) in toluene-d₈ (ca. 0.5 ml) in an NMR tube was one equivalent of ligand 3 (15.9 mg, 0.030 mmol). After 1 h, the reaction was monitored by ¹H NMR spectroscopy which indicated a complete conversion of the yttrium precursor and of the starting ligand with release of 2 equivalents of free amine HN(SiHMe₂)₂. Due to the fluxional behaviour, at ambient temperature, of the product formed, NMR data could only be obtained at 60° C. ¹H NMR characteristic peaks (500 MHz, toluene-d₈, 60° C.): δ main species 7.48 (d, ⁴J(H,H)=2.7 Hz, 4H; aryl), 6.88 (br s, 4H; aryl), 5.00 (m, 1H; SiH(CH₃)₂); secondary species 7.49 (d, ⁴J(H,H)=3.2 Hz, 4H; aryl), 6.81 (d, ⁴J(H,H)=3.2 Hz, 4H; aryl), 4.90 (m, 1H; SiH(CH₃)₂).

### Synthesis of Complex 13.

The yttrium amide Y(N(SiHMe₂)₂)₃.THF₂ (20 mg; 0.033 mmol) was dissolved in deuterated benzene (2 ml) in an NMR tube, then one equivalent of ligand 4 (20.4 mg; 0.033 mmol) was added. The solution was left overnight at ambient temperature. ¹H NMR (C₆D₆, 300 MHz): δ 7.23 (d, ⁴J(H,H)=2.2 Hz, 2H; ArH), 6.18 (d, ⁴J(H,H)=2.4 Hz, 2H; ArH), 5.21 (m, 2H; SiH(CH₃)₂), 3.93 (m, 4H; α-CH₂ THF), 3.74 (d, ²J(H,H)=11.9 Hz, 2H; ArCH₂), 2.98 (d, ²J(H,H)=12.4 Hz, 2H; ArCH₂), 2.84 (t, ³J(H,H)=4.8 Hz, 4H; NCH₂CH₂O), 2.46 (s, 12H; adamantyl), 2.40 (s, 6H; CH₃), 2.26 (s, 9H; adamantyl+OCH₃), 2.03 (d, ²J(H,H)=11.7 Hz, 6H; adamantyl), 1.92 (d, ²J(H,H)=11.3 Hz, 6H; adamantyl), 1.23 (m, 4H; α-CH₂ THF), 0.49 (d, ⁴J(H,H)=2.9 Hz, 12H; SiH(CH₃)₂); ¹³C{¹H} NMR (75 MHz, benzene-d₆, 25° C.): δ 162.39, 129.78, 128.51 (Ar), 72.41 (NCH₂CH₂O), 69.60 (α-CH₂ THF), 63.38 (CH₂Ar), 58.43 NCH₂CH₂O), 48.35 (OCH₃), 40.64 (adamantyl), 37.40 (adamantyl), 37.06 (adamantyl), 29.09 (adamantyl), 24.36 (α-CH₂ THF), 20.33 (CH₃), 3.46 (Si(CH₃)₂); elemental analysis (%) calculated for C₄₇H₇₃N₂O₄Si₂Y: C, 64.50; H, 8.41; N, 3.20. found: C, 64.72; H, 8.27; N, 3.18.

### Synthesis of the [L4LaN(SiHMe₂)₂] Complex 14 (Scheme 2)

The lanthanum amide La(N(SiHMe₂)₂)₃THF₂ (10 mg; 0.016 mmol) was dissolved in deuterated benzene (2 ml) in an NMR tube, then one equivalent of ligand 4 (10.5 mg; 0.016 mmol) was added. The solution was left overnight at ambient temperature. ¹H NMR (C₆D₆, 500 MHz): δ 7.20 (br s, 2H; ArH), 6.80 (br s, 2H; ArH), 5.34 (m, 2H; SiH(CH₃)₂), 3.69 (m, 4H; α-CH₂ THF), 3.38 (m, 2H; ArCH₂), 3.17 (s, 4H; NCH₂CH₂O), 2.82 (m, 2H; ArCH₂), 2.43 (s, 12H; adamantyl), 2.40 (s, 6H; CH₃), 2.19 (s, 9H; adamantyl+OCH₃), 1.99 (d, ²J(H,H)=12.8 Hz, 6H; adamantyl), 1.91 (d, ²J(H,H)=12.8 Hz, 6H; adamantyl), 1.36 (m, 4H; (α-CH₂ THF), 0.49 (d, ⁴J(H,H)=2.9 Hz, 12H; SiH(CH₃)₂); ¹³C{¹H} NMR (125 MHz, benzene-d₆, 25° C.): δ 162.30, 129.70, 128.00 (Ar), 71.29 (CH₂Ar), 69.08 (α-CH₂ THF), 61.48 (NCH₂CH₂O), 48.35 (OCH₃), 40.83 (adamantyl), 37.64 (adamantyl), 37.12 (adamantyl), 29.75 (adamantyl), 25.36 (β-CH₂ THF), 20.98 (CH₃), 3.44 (Si(CH₃)₂).

### Synthesis of Complex 15

A solution of complex 4 (9.3 mg, 0.016 mmol) in benzene (1 ml) was added to a solution of Nd[N(SiMe₃)₂]₃ (10.0 mg, 0.016 mmol) in benzene (1.5 ml) at ambient temperature. The solution was stirred for 12 h at ambient temperature, then 2 h at 60° C. The volatile compounds were then removed under vacuum, then the residue was washed with a minimum amount of cold pentane and dried under vacuum to give 15 in the form of a light blue powder. The product was directly used in polymerization.

### Synthesis of Complex 16

The yttrium amide Y(N(SiHMe₂)₂)₃-THF₂ (10 mg; 0.016 mmol) was dissolved in deuterated benzene (2 ml) in an NMR tube, then one equivalent of ligand 5 (10.5 mg; 0.016 mmol) was added. The solution was left overnight at ambient temperature. ¹H NMR (C₆D₆, 500 MHz): δ 7.54 (d, ⁴J(H,H)=2.3 Hz, 2H; ArH), 7.08 (d, ⁴J(H,H)=2.2 Hz, 2H; ArH), 5.20 (m, 2H; SiHMe₂), 3.82 (m, 4H; α-CH₂ THF), 3.72 (br s, 2H; ArCH₂), 3.11 (d, ²J(H,H)=12.4 Hz, 2H; ArCH₂), 2.82 (t, ³J(H,H)=4.5 Hz, 4H; (NCH₂CH₂O), 2.53 (s, 12H; adamantyl), 2.34 (s, 3H; OCH₃), 2.28 (s, 6H; adamantyl), 2.08 (d, ²J(H,H)=9.2 Hz, 6H; adamantyl), 1.92 (d, ²J(H,H)=10.9 Hz, 6H; adamantyl), 1.47 (s, 18H; C(CH₃)₃), 1.23 (m, 4H; THF), 0.49 (d, ⁴J(H,H)=2.9 Hz, 12H; SiH(CH₃)₂); ¹³C{¹H} NMR (75 MHz, benzene-d₆, 25° C.): δ 162.39, 125.51, 124.21 (Ar), 72.41 (NCH₂CH₂O), 69.70 (α-CH₂ THF), 63.45 (CH₂Ar), 48.35 (OCH₃), 41.14 (adamantyl), 37.57 (adamantyl), 37.48 (adamantyl), 32.42 (C(CH₃)₃), 29.67 (adamantyl), 24.36 (0-CH₂ THF), 4.12 (Si(CH₃)₂); elemental analysis (%) calculated for C₅₃H₈₅N₂O₄Si₂Y: C, 66.36; H, 8.93; N, 2.92. found: C, 66.24; H, 8.73; N, 2.57.

### Synthesis of Complex 17

The yttrium amide Y (N(SiHMe₂)₂)₃.THF₂ (82.6 mg; 0.13 mmol) was dissolved in pentane (5 ml) in a Schlenk tube, then one equivalent of ligand 6 (100 mg; 0.13 mmol) was added in solution in toluene (5 ml). The solution was left overnight at ambient temperature. The solvent was evaporated under vacuum and 95 mg of a white solid were obtained (yield 68%). ¹H NMR (C₆D₆, 500 MHz): δ 7.53 (br s, 2H; ArH), 7.41 (d, ³J(H,H)=7.1 Hz, 8H; cumyl), 7.22 (t, ³J(H,H)=7.7 Hz, 4H; cumyl), 7.13 (m, 6H; cumyl), 6.95 (t, ³J(H,H)=4.5 Hz, 2H; cumyl), 6.78 (br s, 2H, ArH), 4.80 (br s, 2H; SiH(CH₃)₂), 3.35 (br s, 2H; ArCH₂), 2.93 (m, 4H; THF), 2.80 (br s, 3H; OCH₃), 2.69 (br s, 2H; ArCH₂), 2.48 (br s, 2H; NCH₂CH₂O), 2.16 (br s, 6H; CH₃ cumyl), 2.04 (br s, 2H; NCH₂CH₂O), 1.77 (br s, 12H; CH₃ cumyl), 1.74 (br s, 6H; CH₃ cumyl), 1.10 (m, 4H; THF), 0.48 (br s, 12H; SiH(CH₃)₂); ¹³C{¹H} NMR (75 MHz, benzene-d₆, 25° C.): δ 161.34 (Cq, Ar), 152.28 (Cq, cumyl), 137.64, 135.79 (cumyl-C), 128.11, 127.17 (Ar), 127.17, 126.21 (cumyl-C), 72.65 (NCH₂CH₂O), 69.70 (α-CH₂ THF), 63.81 (CH₂Ar), 59.87 (OCH₃), 47.14 (NCH₂CH₂O), 42.49 (C(CH₃)₂), 31.46 (C(CH₃)₂), 27.58 (C(CH₃)₂), 24.81 (β-CH₂ THF), 4.40 (Si(CH₃)₂); elemental analysis (%) calculated for C₆₁H₈₁N₂O₄Si₂Y: C, 69.68; H, 7.77; N, 2.66. found: C, 69.57; H, 7.83; N, 2.74.

### Synthesis of Complex 18 in an NMR Tube

One equivalent of dry isopropanol (0.67 μl, 0.0152 mmol) was added using a microsyringe to a solution of complex 17 (16.0 mg, 0.0152 mmol) in THF-d₈ in an NMR tube. The tube was vigorously shaken and left at ambient temperature for around ten minutes. The reaction was monitored by ¹H NMR, which indicated the complete formation of complex 18. ¹H NMR (THF-d₈, 500 MHz): δ 7.25 (m, 4H; cumyl), 7.20 (m, 8H; cumyl), 7.06 (m, 8H; cumyl), 6.92 (br s, 2H; ArH), 6.69 (br s, 2H; ArH), 4.20 (br m, 1H; CH(CH₃)₂), 3.71 (d, ²J(H,H)=12.0 Hz, 2H; ArCH₂), 3.58 (m, 4H; THF), 3.40 (br s, 3H; OCH₃), 3.01 (br s, 2H; NCH₂CH₂O), 2.86 (d, ²J(H,H)=12.0 Hz, 2H; ArCH₂), 2.16 (br s, 2H; NCH₂CH₂O), 2.04 (br s, 6H; CH₃ cumyl), 1.77 (m, 4H; THF), 1.60 (br s, 12H; CH₃ cumyl), 1.42 (s, 6H; CH₃ cumyl), 1.15 (d, ³J(H,H)=5.9 Hz, 6H; CH(CH₃)₂); ¹³C{¹H} NMR (75 MHz, THF-d₈, 25° C.): δ 162.14 (Cq Ar), 152.07 (Cq cumyl), 134.36 (cumyl-C), 127.38, 126.55, 125.70, 125.08, 123.46 (Ar), 72.77 (NCH₂CH₂O), 69.70 (α-CH₂ THF), 65.63 (CH(CH₃)₂) 63.72 (CH₂Ar), 61.15 (OCH₃), 49.75 (NCH₂CH₂O), 32.37 (C(CH₃)₂), 30.78 (C(CH₃)₂), 28.03 (CH(CH₃)₂), 26.27 (C(CH₃)₂), 24.81 (β-CH₂ THF).

The complexes of the formula (II) mentioned above prepared as is described below and, in particular, complex (18), are active initiators for the ring-opening polymerization of racemic BBL.

Described below is a typical way of carrying out the polymerization of β-butyrolactone.

The complex such as complex 18 is dissolved in toluene (0.6 ml) or in THF (0.6 ml), then 200 equivalents of rac-β-butyrolactone are added using a syringe. The mixture is left stirring at ambient temperature, and the formation of a gel is rapidly observed. The conversion is monitored by NMR. The reaction is terminated by addition of a few drops of an acidified methanol solution (10 vol % HCl solution), then dissolving in chloroform (10 ml). The polymer is precipitated into methanol (100 ml) at ambient temperature. The solution is filtered and the product is dried under vacuum (cf. table).

Representative polymerization data is furthermore collated in Table 2.

The polymerization of racemic BBL with the group 3 metal complex (I) generally takes place rapidly at 20° C.

Some of the polymers produced have narrow molecular weight distributions and number-average molecular weight values (Mₙ) close to theoretical values (calculated by assuming that each isoproxy group initiates the polymerization).

This data indicates that the polymerization takes place in a living manner, that is to say without significant side reactions.

Many samples of PHB are completely insoluble in THF. These samples are, however, readily dissolved, at ambient temperature, in chlorinated solvents such as dichloromethane or chloroform.

A strong influence of the solvent was first observed, including polymerizations in toluene, THF and chlorobenzene.

For a [BBL]/[Y] ratio, Y denoting the complex, especially an yttrium complex, the polymerization in toluene and chlorobenzene achieved a conversion of 97% in less than a minute (Examples 1 and 3), whereas the polymerization in THF achieved a conversion of 98% in the space of 2 hours (Example 4).

Consequently, all the other polymerization reactions were carried out in toluene.

By using a β-diiminate zinc alkoxide initiator under the same reaction conditions, Coates et al. [13] indicated that they succeeded in carrying out the ring-opening polymerization of 200 equivalents of racemic BBL in the space of one hour.

Several reactions were carried out with the yttrium complex (III), modifying the monomer to metal ratio. For example, the complete conversion of 400 equivalents of racemic BBL was obtained in 1 minute at ambient temperature (rotational speed of the catalyst, TOF=24 000 h⁻¹; Example 5).

It should, in particular, be noted that the yttrium alcoholate (isopropylate) (IIl) can polymerize 1000 equivalents in 5 minutes at 20° C. in pure butyrolactone (TOF=12 000 h⁻¹; Example 14) and 2000 equivalents at high concentration in less than 20 minutes (TOF=6000 h⁻¹; Example 15).

Furthermore, the polymerization experiments with complex 1 in the presence of three equivalents of isopropanol show that an alcoholate/isopropanol exchange occurs during the chain propagation process.

After consumption of one equivalent of isopropanol to form the yttrium alcoholate, the excess free alcohol replaces the growing alcoholate polymer chains and acts as a chain transfer agent (Example 10).

An important objective which is pursued in the present invention is to study the asymmetric incorporation of BBL into the main chain of the polymer.

For these purposes, ¹³C NMR spectroscopy can be used to determine the stereochemistry of the PHBs by inspecting the carbonyl region (for “diads”) and the methylene region (for “triads”) of the ¹³C NMR spectra of the polymers.

The microstructural analysis of the various PHBs formed from the racemic BBL reveals that the group 3 metal complex (II) exerts, at ambient temperature, a significant influence on the tacticity of the polymer formed according to scheme 2 below:

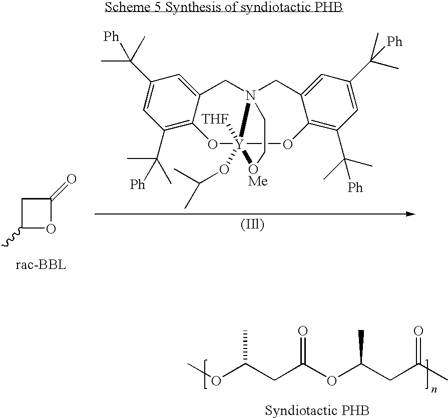

Based on the preceding attributions of the ¹³C NMR peaks of PHB, the upfield and downfield signals of the carbonyl region correspond respectively to the meso(m) diad blocks (R—R) and (S—S) and to the racemic(r) diad blocks (R—S) and (S—R).

It is interesting to note that two stereochemical blocks (r) are different because of the “directionality” effect of the ester bond, and are clearly separated.

The PHBs prepared by the method of the invention reveal a strong contribution of the r diads (δ 169.15 ppm) which are evidence of a PHB highly enriched in the syndiotactic form (FIG. 1a).

Up to 94% of r diads (Pᵣ=0.94) were detected depending on the reaction conditions used.

The observation of the expansion of the methylene region in FIG. 1b shows three peaks (the fourth theoretical peak is not intense enough to be observed), which correspond to the triad sensitivity.

Based on the preceding attributions, it was determined that the most intense resonance at 40.80 ppm corresponds to the (rr) triad with a relative integration of 86%. It should be noted that the separation of the two stereochemical blocks has been observed during the expansion of the regions of the signals of the methyl carbons (separated into two peaks) and methine carbons (separated into three peaks).

Among the signals of the methyl and methine carbons, the diad and triad blocks have been attributed and are in good agreement with the signals of the carbonyl and methylene carbons.

Differences have been observed in the microstructure of PHB as a function of the nature of the solvent used.

Whereas the polymerization of racemic BBL in toluene and chlorobenzene gives a PHB with close to 90% of syndiotactic linkages (Pᵣ=0.90), the polymerization in THF gives only 83% syndiotacticity (Pᵣ=0.83).

We are also interested in the influence of the temperature. It is obvious that the degree of syndiotactic stereoregularity decreases gradually as the polymerization temperature increases as was anticipated.

At low temperatures (−20° C.) the polymerization takes place slowly, but the PHB obtained has a high degree of syndiotacticity (Pᵣ=0.94; Example 8). On the other hand, at higher temperatures the polycondensation takes place more rapidly (800 equivalents converted in 3 minutes at 50° C. instead of 10 minutes at ambient temperature), as could be anticipated, and provides PHBs with only 85% syndiotactic linkages and probably with a broader polydispersity (Examples 11 and 12).

The thermal analyses of certain samples were carried out and reveal the strong influence of the stereochemistry on the physical properties of the PHB.

It appears, by observing FIG. 2, that the samples of polymers synthesized with the yttrium complex 1 have a single narrow transition endotherm, thus forming a uniform crystalline arrangement in the solid state, which may indicate a narrower dispersion of the stereoblocks of the chain (for Pᵣ=0.85, Tₘ=138° C. and for Pᵣ=0.91, Tₘ=165° C.). This result is very different from the previous descriptions given in the literature, which describe two melting endotherms [15] or even three melting endotherms [16].

TABLE 2
Polymerization of β-butyrolactone with complex 1^{a}

| Example | [BBL]/[1] | [BBL] (mol/l) | Solvent | Temperature (° C.) | Time (min.) | Conversion (%)^{b} | Mₙ^{c} (g/mol) | PDI^{c} | Pᵣ^{d} |
|---|---|---|---|---|---|---|---|---|---|
| 1 | 200 | 2.44 | Toluene | 25 | 1 | 98 | — | — | 0.88 |
| 2 | 200 | 2.44 | Toluene | −20 | 10 | 95 | — | — | 0.91 |
| 3 | 200 | 2.44 | Chlorobenzene | 25 | 1 | 95 | — | — | — |
| 4 | 200 | 2.44 | THF | 25 | 120 | 98 | — | — | 0.83 |
| 5 | 400 | 2.44 | Toluene | 25 | 1 | 98 | 26100 | 1.06 | 0.89 |
| 6 | 400 | 1.00 | Toluene | 25 | 10 | 98 | — | — | 0.90 |
| 7 | 400 | 4.80 | Toluene | 25 | 4 | 95 | — | — | 0.87 |
| 8 | 400 | 1.00 | Toluene | −20 | — | 98 | — | — | 0.94 |
| 9 | 600 | 2.44 | Toluene | 25 | 5 | 98 | 47200 | 1.10 | — |
| 10^{e} | 800 | 2.44 | Toluene | 25 | 8 | 97 | 21900 | 1.17 | 0.90 |
| 11 | 800 | 2.44 | Toluene | 25 | 10 | 95 | — | — | — |
| 12 | 800 | 2.44 | Toluene | 50 | 3 | 96 | — | — | 0.85 |
| 13 | 800 | 2.44 | Toluene | −20 | — | 38 | — | — | 0.92 |
| 14 | 1000 | Neat | — | 25 | 5 | 95 | — | — | — |
| 15 | 2000 | 8.80 | Toluene | 25 | 18 | 87 | — | — | — |

^{a}All the reactions were carried out with complex 1.
^{b}Such as determined by the integration of the methine resonances of BBL and of PHB in H NMR.
^{c}Mₙ of the H and M_{w}/Mₙ of the polymer determined by SEC-IR in THF at ambient temperature, using polystyrene standards.
^{d}Pᵣ is the probability of racemic bonds between the monomer units and is determined from the carbonyl region of the ¹³C spectrum with three equivalents of isopropanol.

### REFERENCES

[1] Drumright, R. E.; Gruber, P. R.; Henton, D. E. Adv. Mater. 2000, 12, 1841.
[2] Senior, P. J. and Dawes, E. A. Biochem. J. 1973, 134, 225-238.
[3] (a) Müller, H.-M.; Seebach, D. Angew. Chem. Int. Ed. Engl. 1993, 32, 477-502. (b) Sudesh, K.; Abe, H.; Doi, Y. Progr. Polym. Sci. 2000, 25, 1503-1555.
[4] (a) Doi, Y.; Kumagai, Y.; Tanahashi, N.; Mukai, K. In Biodegradable Polymers and Plastics; Vert, M., Feijen, J., Albertsson, A., Scott, G., Chiellini, E., Eds.; The Royal Society of Chemistry: Cambridge, 1992; pp 139-148. (b) Cox, M. K. In Biodegradable Polymers and Plastics; Vert, M., Feijen, J., Albertsson, A., Scott, G., Chiellini, E., Eds.; The Royal Society of Chemistry: Cambridge, 1992; pp 95-99. (c) Okada, M. Progr. Polym. Sci. 2002, 27, 87-133.
[5] Holmes, P. A. Phys. Technol. 1985, 16, 32-36.
[6] Ritter, S. K. Chemical & Engineering News 2005, 83(26), June 27.
[7] Anderson, A. J., Dawes, E. A., Microbiol. Rev. 1990, 54, 450.
[8] Okada, M. Prog. Polym. Sci. 2002, 27, 87-133.

[9] Yori, Y., Suzuki, M., Yamaguchi, A., Nishishita, T. Macromolecules, 1993, 26, 5533.
[10] (a) Billingham, N. C.; Proctor, M. G.; Smith, J. D. J. Organomet. Chem. 1988, 341, 83. (b) Moeller, M.; Kånge, R.; Hedrick, J. L. J. Polym. Sci., Part A 2000, 38, 2067. (c) Schechtman, L. A.; Kemper, J. J. PCT Int. Appl., WO 0077072, 2000.

[11] (a) Wu, B.; Lenz, R. W. Macromolecules 1998, 31, 3473. (b) Lenz, R. W.; Yang, J.; Wu, B.; Harlan, C. J.; Barron, A. R. Can. J. Microbiol. 1995, 41, 274. (C) Bloembergen, S.; Holden, D. A.; Bluhm, T. L.; Hamer, G. K.; Marchessault, R. H. Macromolecules 1989, 22, 1656.

[12] (a) Hori, Y.; Hagiwara, T. Int. J. Biol. Macromol. 1999, 25, 237. (b) Kricheldorf, H. R.; Eggerstedt, S. Macromolecules 1997, 30, 5693.

[13] Rieth, L. R., Moore, D. R., Lobkovsky, E. B., Coates, G. W. J. Am. Chem. Soc. 2002, 124, 15239.

[14] (a) Cai, C.-X.; Amgoune, A.; Lehmann, C. W.; J.-F. Carpentier Chem. Commun. 2004, 330-331. (b) Amgoune, A.; Thomas, C. M.; Balnois, E.; Grohens, Y.; Lutz, P. J.; Carpentier J.-F. Macromol. Rapid Commun. 2005, 26, 1145-1150.

[15] (a) Kricheldorf, H. R., Lee, S—R., Scharnagi, N. Macromolecules 1994, 27, 3139. (b) Gross, R. A., Zhang, Y., Konrad, G., Lenz, R. W., Macromolecules 1988, 21, 2657.

[16] Kemnitzer, J. E., McCarthy, S. P., Gross, R. A. Macromolecules 1993, 26, 1221.

1. Method for preparing a polyhydroxyalkanoate (PHA) polymer by ring-opening polymerization of a lactone of formula (I): 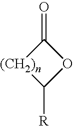
where n is an integer from 1 to 4, and R represents a hydrogen or a linear or branched C₁₋₄ alkyl group, characterized in that the polymerization is carried out in the presence of an initiator of formula (II): 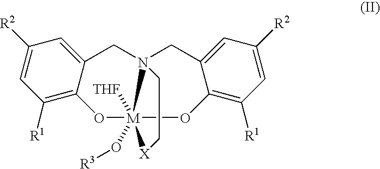
in which:
R³ represents a C₁₋₁₅ alkyl group such as a methyl, ethyl, isopropyl, n-propyl, n-butyl, isobutyl or tert-butyl group; or a benzyl group;
R¹ and R², being identical or different, each represent a group chosen from C₁₋₁₅ alkyl groups, such as methyl, tert-butyl groups; a cumyl group; an α,α-dimethylbenzyl group; an adamantyl group; a trityl group; and a (C₃ to C₁₅)trialkylsilyl group;
X represents O(R₄), S(R₄) or N(R₄)(R₅) where R₄ and R₅ each independently represent a C₁₋₁₅ alkyl group such as a methyl or ethyl group; or a benzyl group;
M is a metal from group 3 of the Periodic Table of the Elements such as Y, La or Nd.

2. Method according to claim 1, in which the lactone is chosen from the lactones corresponding to the following formulae: 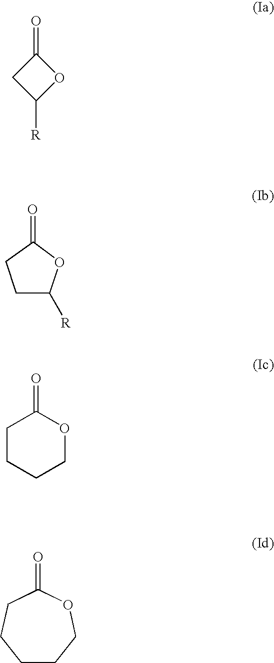
in which R has the meaning already given in claim 1, and, preferably, in the formula (Ia), R represents H, or a methyl or ethyl group and in the formula (Ib), R represents H, or a methyl, ethyl or propyl group.

3. Method according to either one of the preceding claims, in which the lactone (I) is a racemic lactone and the polyhydroxyalkanoate (PHA) prepared is a syndiotactic PHA.

4. Method according to claim 3, in which the lactone is racemic β-butyrolactone or racemic γ-valerolactone.

5. Method according to either one of claims 3 and 4, in which the PHA prepared has a degree of syndiotacticity Pᵣ of 70 to 99%.

6. Method according to any one of the preceding claims, in which the initiator of formula (II) is chosen from the compounds of formulae below: 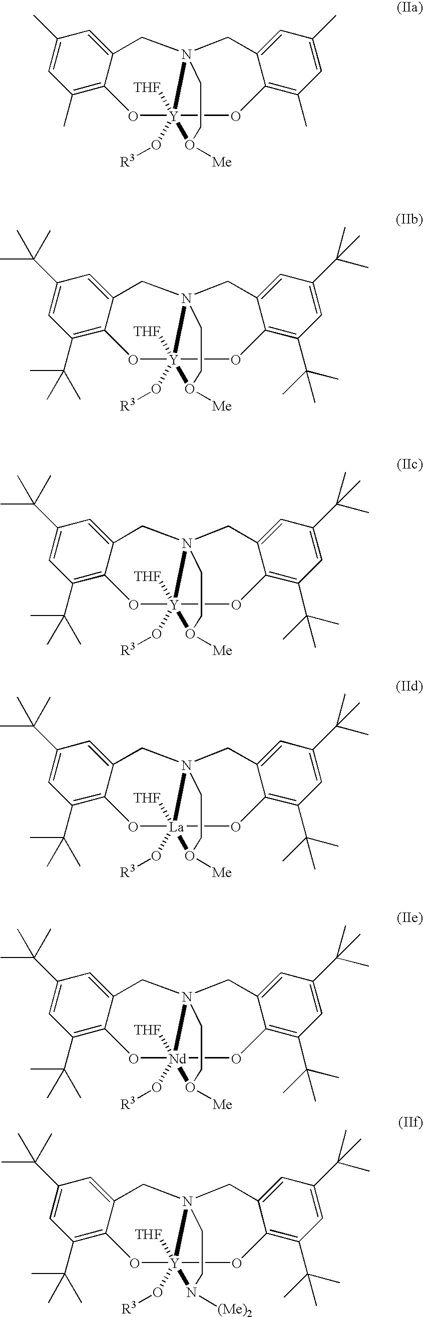 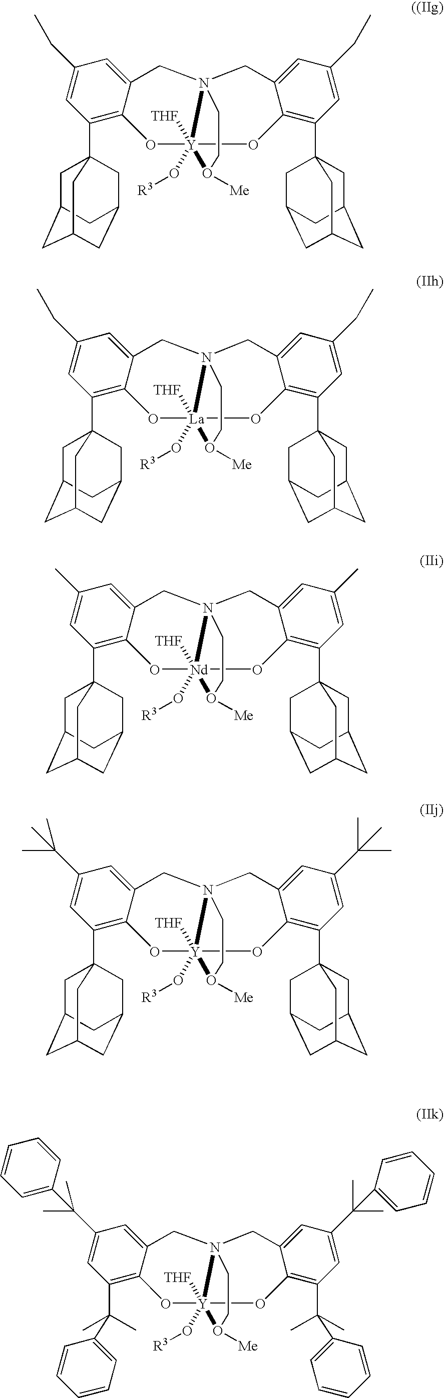
in which R³ has the meaning already given in claim 1 and preferably represents an isopropyl group.

7. Method according to any one of the preceding claims, in which the initiator of formula (II) is the following compound:

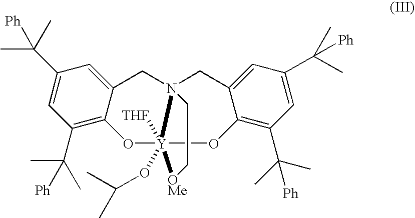

8. Method according to any one of the preceding claims, in which the polymerization is carried out in a solvent chosen from toluene, tetrahydrofuran, chlorobenzene and mixtures thereof.

9. Method according to any one of the preceding claims, in which the ratio of the lactone concentration to that of the initiator is from 50 to 5000, preferably from 100 to 2000.

10. Method according to any one of the preceding claims, in which the polymerization is carried out at a temperature from −30 to 120° C., preferably from 0 to 60° C., more preferably from 15 to 30° C., for example at 20° C.

11. Method according to any one of the preceding claims, in which the lactone is racemic β-butyro-lactone, the complex is the complex of formula (III) according to claim 5, the polymerization is carried out in toluene at a temperature from −30 to 110° C., preferably from 0 to 60° C., and the ratio of the β-butyrolactone concentration to that of the initiator is from 100 to 2000.

12. Method according to any one of the preceding claims, in which the polymer prepared has a number-average molecular weight of 4000 to 500 000, preferably from 8000 or 9000 to 170 000.

13. Method according to any one of the preceding claims, in which the polymer prepared has a polydispersity index PDI from 1.01 to 1.50, preferably from 1.05 to 1.20.

14. Polyhydroxyalkanoate polymer obtainable by the method according to any one of claims 1 to 13.

15. Polymer according to claim 14, in which the polyhydroxyalkanoate is a syndiotactic PHA obtainable by polymerization of a racemic lactone (I).

16. Polymer according to claim 15, in which the lactone is racemic β-butyrolactone or racemic γ-valero-lactone.

17. Polymer according to any one of claims 15 and 16, which has a degree of syndiotacticity of 70 to 99%.

18. Polymer according to any one of claims 14 to 17, which has a number-average molecular weight of 4000 to 500 000, preferably from 8000 or 9000 to 170 000.

19. Polymer according to any one of claims 14 to 18, which has a polydispersity index PDI from 1.01 to 1.50, preferably from 1.05 to 1.20.

20. Material composition comprising the polyhydroxy-alkanoate polymer according to any one of claims 14 to 19 and other ingredients.

21. Composition according to claim 20, characterized in that it is biodegradable.

22. Composition according to claim 21, comprising, in addition, one or more biodegradable polymers different from the polyhydroxyalkanoate.

23. Composition according to any one of claims 20 to 23, in which the polyhydroxyalkanoate polymer is present in an amount of 0.1 to 99.9%, preferably 1 to 99%, more preferably 5 to 90%, better 10 to 80%, better still 20 to 70%, for example 40 to 60%, especially 50% or 55% by weight of the total weight of the composition.

24. Use of the polymer according to any one of claims 14 to 19 or of the composition according to any one of claims 20 to 23, in packaging, especially food packaging.

25. Use of the polymer according to any one of claims 14 to 19 or of the composition according to any one of claims 20 to 23, in biomedical devices and processes or for biomedical applications.

26. Use of the polymer according to any one of claims 14 to 19 or of the composition according to any one of claims 20 to 23, in surgical fasteners such as sutures, agrafes and plates.

27. Use of the polymer according to any one of claims 14 to 19 or of the composition according to any one of claims 20 to 23, for the controlled, delayed, release of medicaments.
